(19) United States
(12) Patent Application Publication
(10) Pub. No.: US 2014/0276756 A1
(43) Pub. Date: Sep. 18, 2014

(54) WALL-SPARING RENAL NERVE ABLATION CATHETER WITH SPACED ELECTRODE STRUCTURES

(71) Applicant: Boston Scientific Scimed, Inc., Maple Grove, MN (US)

(72) Inventor: JASON P. HILL, BROOKLYN PARK, MN (US)

(73) Assignee: BOSTON SCIENTIFIC SCIMED, INC., MAPLE GROVE, MN (US)

(21) Appl. No.: 14/196,588

(22) Filed: Mar. 4, 2014

Related U.S. Application Data
(60) Provisional application No. 61/792,993, filed on Mar. 15, 2013.

(51) Int. Cl. A61B 18/18

(52) U.S. Cl. CPC A61B 18/18
USPC 606/33

(57) ABSTRACT

Systems and methods for performing tissue modulation are disclosed. A nerve modulation assembly may include a guide sheath having a proximal end, a distal end, and a lumen extending therebetween. An actuation member, also having a proximal end and a distal end, extends along a central elongate axis disposed within the lumen. Further, the ablation member disposed at the distal end of the actuation member is configured to be actuated from the proximal end of the guide sheath. In particular, the ablation member includes a radiofrequency based electrode member, and an insulation member disposed circumferentially around the radiofrequency based electrode member.

### CROSS-REFERENCE TO RELATED APPLICATIONS

This application claims priority under 35 U.S.C. §119 to U.S. Provisional Application Ser. No. 61/792,993, filed Mar. 15, 2013, the entirety of which is incorporated herein by reference.

### TECHNICAL FIELD

This disclosure relates to devices and methods for intravascular neuromodulation. More particularly, the technologies disclosed herein relate to apparatus, systems, and methods, for achieving intravascular renal neuromodulation via an ablation member that includes one or more spacing members to position an electrode away from an intravascular wall.

### BACKGROUND

Certain treatments require temporary or permanent interruption, or modification of select nerve functions. Such exemplary nerve functions may include treatments to regions around a kidney. One such treatment is renal nerve ablation, which at times is used to treat a condition related to a congestive heart failure. In this condition, the kidneys produce a sympathetic response to a condition of a congestive heart failure, which among other effects, increases the undesired retention of water and/or sodium. Ablating some nerves running to the kidneys may reduce or eliminate this sympathetic response, providing a corresponding reduction in the associated undesired symptoms. For example, a renal nerve ablation procedure is often used to lower the blood pressure of hypertensive patients.

Many nerves, including renal nerves, run along the walls of or in close proximity to blood vessels, and these nerves may be accessed intravascularly through blood vessels. In some instances, it may be desirable to ablate or otherwise modulate perivascular nerves such as renal nerves using energy such as that provided by radio frequency (RF) electrodes, ultrasonic elements and the like. Such treatment, however, may result in thermal injury to the vessel at the electrode and other undesirable side effects such as, but not limited to, blood damage, clotting, and/or protein fouling of the electrode. To prevent such undesirable side effects, some techniques attempt to increase the distance between the vessel walls and the electrode. In these systems, however, the electrode may inadvertently contact the vessel walls, causing undesirable damage. Thus, there remains an ongoing need for alternative devices and methods.

### SUMMARY

The disclosure is directed to several alternative designs, materials, and methods of manufacturing medical device structures and assemblies.

Accordingly, some illustrative embodiments pertain to a nerve modulation assembly designed for percutaneous operation such as intravascular operation. The assembly includes a guide sheath having a proximal end, a distal end, and a lumen extending therebetween. An elongate device, including a proximal end and a distal end as well, extends within the lumen of the guide sheath. Further, an ablation member disposed at the distal end of the elongate device may include one or more ablation elements, such as a radio frequency (RF) based electrode member, and one or more spacing members, disposed circumferentially around the one or more ablation elements. The spacing members may be configured to keep the one or more ablation elements from contact with the vessel wall during operation. The ablation member is configured to be deployed from the distal end of the guide sheath. More so, operation of the ablation member, through the elongate device, is enabled through an external control unit operably connected through the proximal end of the elongate device. In some embodiments, the ablation elements, including one or more RF electrode members, may be disposed on a balloon or may be a slotted-tube electrode or the like. In some embodiments, the one or more spacing members may be a nonconductive stent-like member or one or more of a series of nonconductive bumps on a balloon. The nonconductive stent-like member may be a braid member that includes a plurality of loosely braided spiral coils, an expandable tube formed into a network of connected struts or the like. In some embodiments, the ablation member is configured to switch between a collapsed position for delivery and/or withdrawal and an expanded position for operation. In the expanded position, the one or more spacing members may contact the inner wall to keep the electrode member at a distance from the inner wall. In some embodiments, moving the ablation element between the expanded and collapsed positions may be effected through a relative movement between the elongate device and the guide sheath. In another embodiment, moving the ablation element between the expanded and collapsed positions may be effected through use of an actuation element such as a pull wire or the introduction or withdrawal of an inflation medium.

Another illustrative embodiment pertains to a method for ablating a nerve through a blood vessel. The method includes providing an ablation system including a guide sheath, having a proximal end and a distal end, and having an ablation member disposed at the distal end of an elongate device extending within the guide sheath. Herein, the ablation member includes a RF based electrode member, and a spacing member disposed circumferentially around the electrode member. In particular, the ablation member includes one or more RF electrode members and may be disposed on a balloon or may be a slotted-tube electrode or the like, while the spacing member may be a nonconductive stent-like member or one or more of a series of nonconductive bumps on a balloon. The ablation system may be advanced to a desired location within the blood vessel. The method further includes deploying the ablation member at the desired location within the blood vessel and using the ablation member such that portions of the spacing member contact the blood vessel's inner wall, while enabling the RF based electrode member to avoid direct contact with the inner wall. Further, the method includes activating the electrode member to ablate a desired portion adjacent to the blood vessel through a RF based control unit disposed at the proximal end of the elongate device. Moreover, deploying the ablation member includes switching the ablation member from a collapsed position to an expanded position, such that a portion of the spacing member contacts the inner wall of the blood vessel in the expanded position, while keeping the electrode member at a distance from the inner wall.

The summary is not intended to describe each disclosed embodiment or every implementation of the disclosure.

### BRIEF DESCRIPTION OF THE DRAWINGS

The present disclosure may be more completely understood in consideration of the following detailed description of various embodiments in connection with the accompanying drawings, in which:

FIG. 1 is an isometric view of the distal portion of an embodiment of a nerve modulation assembly, according to the present disclosure.

FIG. 2 is an isometric view of another embodiment of a nerve modulation assembly, according to the present disclosure.

FIG. 3A is an isometric view of another embodiment of a nerve modulation assembly, according to the present disclosure.

FIG. 3B is an isometric view of another embodiment of a nerve modulation assembly, according to the present disclosure.

FIG. 4 is one of the embodiments depicted in FIG. 1, 2, or 3, in application within a human body.

FIG. 5 is a detailed view of the embodiment depicted in FIG. 4, while in application within a blood vessel.

While embodiments of the present disclosure are amenable to various modifications and alternative forms, specifics thereof have been shown by way of example in the drawings and will be described in detail. It should be understood, however, that the intention is not to limit aspects of the disclosure to the particular embodiments described. One the contrary, the intention is to cover all modifications, equivalents, and alternatives falling within the spirit and scope of the present disclosure.

### DETAILED DESCRIPTION

For the following defined terms, these definitions shall be applied, unless a different definition is given in the claims or elsewhere in the specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimension ranges and/or values pertaining to various components, features, and/or specifications are disclosed, one of skill in the art, incited by the present disclosure, would understand desired dimensions, ranges and/or values many deviate from those expressly disclosed.

As used in this specification and the appended claims, the singular forms “a,” “an,” and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired. For example, the devices and methods described herein may also be used for prostate ablation, tumor ablation, and/or other therapies requiring heating or ablation of target tissue. Further, while the devices and methods described herein are discussed relative to RF electrodes and RF modulation and ablation techniques, it is contemplated that other ablation technologies may be used in lieu of the RF technologies. For example, electromagnetic frequencies outside the RF range and/or ultrasonic transducers and energy may be used in some embodiments.

In some instances, it may be desirable to ablate perivascular renal nerves with deep target tissue heating. However, as energy passes from an electrode to the desired treatment region, the energy may heat the fluid (e.g. blood) and tissue as it passes. As more energy is used, higher temperatures in the desired treatment region may be achieved, but may result in some negative side effects, such as, but not limited to, thermal injury to the vessel wall, blood damage, clotting, and/or electrode fouling. Positioning the electrode away from the vessel wall may provide some degree of passive cooling by allowing blood to flow past the electrode.

FIG. 1 is an isometric view of an illustrative nerve modulation system or ablation system 130 having a longitudinal axis 122 and including an ablation member 100. The illustrative nerve modulation system 130 may be a part of a nerve modulation assembly referred to as a renal nerve ablation system 400 (depicted in FIG. 4), according to the present disclosure. As illustrated, the ablation member 100 includes a distal end 124 and a proximal end 126. Further, the ablation member 100 is disposed towards a distal end region 128 of an elongate device 113. In some instances, the nerve modulation system 130 may be disposed within a lumen of a catheter sheath, referred to as a guide sheath, such as guide sheath 111 shown in FIG. 4.

Ablation member 100 includes one or more spacing members, which, in the illustrated embodiment, may be a nonconductive expandable braid member 102, made up of loosely braided spiral coils 104. These elements combine to form a generally cylindrical cage structure, dimensioned to bear against the inner walls of a selected blood vessel, positioning the ablation member 100 within the vessel in a selected position, as explained below.

Some embodiments may replace the spiral coils 104 with structures of generally similar construction but different shape. For example, while the braid member 102 and spiral coils 104 are illustrated as having a cylindrical cage structure, other shapes may be used, such as, but not limited to, a generally spherical configuration, a polygonal cross-section or the like. More particularly, any structure suited to position an ablation element within a typical blood vessel and away from the wall of the blood vessel may be incorporated, and accordingly the embodiment shown in FIG. 1 should not be understood as limiting the structural aspects of the disclosure in any way. For example, a spacing member may be provided that that has a stent-like configuration, with a plurality of generally interconnected struts forming an expandable tubular member or may be a multi-ribbon tube.

Electrode members 117 may comprise a number of ribbon-shaped members lying inside the spiral coils 104. Taken together, these elements may be formed a multi-ribbon slotted tube structure, which could also be described as a ribbon-based slotted tube electrode, although this is not required. In some instances, the electrode members 117 may be formed as separate ribbons or filaments. The number of electrode members 117 and their spacing both from each other and from the spacing member can be varied to suit particular applications. Those of skill in the art will be able to resolve such design considerations. As an example, there may be 2, 3, 4, 5, 6, 7, 8 or more of the electrode members 117. In some embodiments, the electrode members 117 may be equally or unequally spaced from each other. In some embodiments, the electrode members 117 may be at the same longitudinal location. In other embodiments, the electrode members 117 may be at different longitudinal locations. For example, the electrodes may form a zig-zag pattern or may form a helical pattern, etc. In some embodiments, each electrode member 117 may include one or more insulated segments 118, and one or more electrode pads 116. In some embodiments, the insulated segments may be located proximal to, distal to, or both proximal and distal to the electrode pads. As can be understood from FIG. 1, the number, size, and design of the insulated segments 118 may vary based on design and application factors. In addition, the number of electrode pads 116 disposed on each electrode member 117 may vary as well. For example, in some instances, there may be more than one electrode pad 116 positioned along one or more of the electrode members 117. Insulated segments 118 may be formed using techniques well known in the art, and accordingly these elements may include insulated coatings. For example, all or portions of the electrode members 117 may be coated with an insulating coating by dip or spray coating, chemical vapor deposition, parylene coating, etc. In some instances, the entire length of the each of electrode members 117 may be coated with an insulating coating with portions removed to define electrode pads 116 and a region for contact with one or more electrical supply wires or conductors. Further, the electrode pads 116 may incorporate a surface area larger than similar elements employed in conventional applications, such as wired electrodes and the like. In some instances, the electrode pads 116 may have a larger width than the remaining portions of the electrode member 117. In addition, the relatively large surface area of the electrode pads 116, when employed, spreads the generated heat over a relatively large surface area, reducing the temperature at any given point on the electrode pads 116, minimizing blood fouling and damage. Furthermore, the “off-the-wall” electrode design minimizes damage to the walls of the blood vessels themselves.

More particularly, each electrode pad 116 may be a flat electrode formed of nitinol, platinum, gold, stainless steel, cobalt alloys, or other suitable materials. In some embodiments, titanium, tantalum, or tungsten may be used as well. It is contemplated that if the material used for the electrode pad 116 is not radiopaque, the electrode pad 116 may be coated with a layer of radiopaque material such as gold or tantalum to allow accurate electrode placement using fluoroscopy. In other embodiments, the electrode member 117 may extend along substantially the entire length of the nonconductive braid member 102. Because the electrode member 117 is a generally ribbon-shaped member, it may, for example, extend helically as well. Each of the electrode members 117 up to at least the electrode pads 116 is conductive, and is electrically connected to the power supply, such as power and control unit 402 (shown in FIG. 4). The control unit 402 may be based on radio frequency control, etc. The electrode members 117 may be connected to the control unit 402 located outside the body as a group by, for example, a common conductor, such as the conductor 404 (shown in FIG. 4), or could be separately connected and controlled. The one or more electrical conductors may be electrically connected to a proximal end of the electrode members 117. However, this is not required. In some instances, the one or more electrical conductors may be directly connected to the electrode pads 116.

In some instances, the ablation system 130 may include an element 114 forming an actuation member or a pull wire, fixed to the distal end 124. The actuation member 114 may be slidable within an elongate shaft 112 of the elongate device 113. This element 114 serves to expand or contract the cage structure upon user actuation, as discussed below. Also, as noted above, element 114 can be locked to the outer shaft 110.

In some embodiments, the braid member 102 may be biased to the expanded position so that it expands when the guide sheath or a retractable sheath that passes through the guide sheath is withdrawn; in such cases, no pull wire or element 114 is required. In some cases, the electrode member 117 may be spirally disposed relative to the element 114, forming a spiral shaped electrode configuration in an expanded position. In other embodiments, electrode member 117 may be a wire, filament, or a tubular member disposed relative to the element 114. In embodiments that include more than one electrode member 117, each electrode member 117 may be separately controllable as well. Further, the electrode member 117 may be selected to provide a particular level of flexibility as well during an application. Such flexibility may be configured in the transverse and/or in the linear plane, and may particularly depend upon a cross sectional area of a blood vessel within which the system 400 is employed for an ablation.

The braid member 102 and electrode members 117 may be generally attached at the proximal end of the ablation member to an elongate member 112. The elongate member 112 may include one or more tubular members 110, 108 and one or more lumens 106. The tubular member 108 may, for example, be the proximal portion of pull wire 114 or may be a hollow tubular member through which the pull wire 114 extends. While member 108 is described as tubular, it is contemplated that in some embodiments, member 108 may have a generally solid cross-section.

It will be readily understood by those in the art that FIG. 1 depicts the ablation member 100 in an expanded configuration. Ablation member 100 may assume a collapsed configuration when carried within the distal end of a guide sheath, such as guide sheath 111 shown in FIG. 4 during the period when system 130 is introduced into a blood vessel and advanced to an ablation site, as described in more detail below. When system 130 is positioned in a desired spot for ablation, the operator expands ablation member 100 by extending it distally from guide sheath 111 by operation of elongate device 113. When the ablation member 100 is biased to the expanded configuration, the guide sheath may maintain the ablation member 100 in the collapsed position and distal actuation of the elongate device 113 to advance the ablation member 100 out of the guide sheath may allow the ablation member 100 to expand. In other instances, once the ablation member 100 has been advanced out of the guide sheath, actuation of the actuation member 114 may be required to expand the ablation member. As ablation member 100 is expanded, end struts 120, positioned at the distal end region of the ablation member 100 may support the braid member 102 and/or electrode members 117 maintaining its overall geometry.

Techniques for such deployment including the expansion and collapse of the ablation member 100 may be well understood by comparing descriptions of FIG. 2 and FIG. 5, showing elongate device 113, extending along the central elongate axis and disposed within a lumen of the vessel 510. In particular, the elongate device 113 may be configured to push and/or pull on the distal end 518 of the elongate device 113 from its proximal end 506 (Shown in FIG. 5), deploying the ablation member 100 at a desired site within the blood vessel. After treatment is complete, the nonconductive braid member 102, including the internal structure of electrode members 117 may be collapsed and returned into the guide sheath 111 for removal from the blood vessel. In particular, the expansion and collapse of the ablation member 100 may be controlled through a relative movement between the elongate device and the guide sheath 111 and/or actuation member 114. In alternate embodiments, the electrode member 117 discussed above may be configured as a spiral or other suitable shape, as may be considered advantageous for particular situations. Accordingly, the embodiments discussed in the present application do no limit the shape and configuration of the electrode member 117 in any way.

FIG. 2 depicts an alternative embodiment of another illustrative ablation system 230 including an ablation member 200, in which a balloon electrode 204 replaces the electrode members 117 shown in FIG. 1. The outer structure and configuration of the ablation member 200 remains generally the same as that seen in ablation member 100 and therefore will not be discussed here. For example, the structure of the expandable braid member 102 and the elongate device 103 may be similar in form and function to the expandable braid member 102 and the elongate device 113 described with respect to FIG. 1, although this is not required. For example, the structure of the elongate device 113 may be modified to provide an inflation lumen in fluid communication with the balloon electrode 204.

Balloon electrode 204, like the electrode member 117, is disposed within the nonconductive braid member 102, the diameter of the balloon electrode 204 being less than the diameter of the nonconductive braid member 102. As was discussed above in connection with ablation member 100, some embodiments of nonconductive braid member 102 may be biased to the expanded configuration, or self-expandable, formed of resilient material that expands to full diameter upon being advanced from a guide sheath. Some embodiments may include a braid member 102 that is expanded by operation of a pull wire, similar to pull wire 114 in FIG. 1, attached to the distal end 124 of the ablation member 200. The balloon electrode 204 may then be expanded separately from the braid member 102, for example, through the delivery of an inflation fluid. In some embodiments, it is possible thereby to control the distance the balloon electrode outer surface is from the braid member 102 when in the expanded position. For such embodiments, balloon electrode 204 may be provided with a lumen (not shown), for example in tubular member 108, through which element 114 extends. In other instance, the balloon electrode 204 may be expanded using an inflation fluid supplied through an inflation lumen in the elongate device 113. Other features related to expansion of the braid member 102 are discussed above or are well known to those of skill in the art and will not be discussed further here.

In some embodiments, balloon electrode 204 may be formed from a compliant (elastic) material. That construction would enable the balloon to inflate to different diameters, which in turn allows the electrodes to be positioned a desired distance from the wall of the blood vessel. For example, a balloon with a nominal diameter of 6 mm may be adjustable to have an operating diameter of between 5 and 7 mm. This allows for some adjustability to accommodate blood vessels of different sizes.

In addition, embodiments of balloon electrode 204 may be provided with several different types of electrodes for accomplishing ablation. Some embodiments may include electroconductive portions, such as a portion 206, on the balloon surface, each portion 206 functioning as an electrode. Those in the art will understand that the electroconductive portions can be designed to produce a desired pattern of energy delivery adaptable to particular situations. For example, in some embodiments, the electroconductive portion 206 may extend over the entire length of the balloon electrode 204 or over only a portion of the balloon electrode 204. In some instances, the electroconductive portion 206 may extend around the entire circumference of the balloon electrode 204 while in other instances the electroconductive portion 206 may extend around only a portion of the circumference of the balloon electrode 204. For example, electroconductive portions 206 may be printed on the surface of balloon electrode 204 in any desired shape or configuration, including leads connecting each portion 206 to a power source (not shown). That ability facilitates shaping the electrodes to accomplish particular purposes in energy delivery to the renal nerves. Moreover, the fact that large portions of the balloon electrode 204 can function as electrodes leads to a low current density, minimizing blood fouling and damage. In some instances, balloon 204 may include an insulated end portion 208 positioned proximally, distally or both proximally and distally of the electroconductive portion 206. Other embodiments of balloon electrode 204 may employ a central electrode located inside the balloon itself, the balloon 204 being filled with an electroconductive fluid. For such embodiments, portions 206 are formed as windows of hydrophilic material in the balloon wall. The electrode may generate energy, such as RF energy, which is then carried through the electro-conductive fluid and transmitted through the windows.

It will be understood that embodiments of the present disclosure are not limited to standalone electrodes, array electrodes, or balloon electrodes of various configurations. These and other electrode structures, now known or later developed, may be employed in embodiments of the present disclosure without departing from the spirit of the invention.

A variety of cooling regimes may be employed to prevent heat buildup. Balloon electrode 204 can be provided with a continuous flow of a liquid or gas to promote heat transfer, thus minimizing blood fouling and damage. Saline, or plain water, can be used as the fluid. Construction and arrangement of fluid flow devices to enable the expansion and contraction of balloon electrode 204 are entirely within the skill of those in the art and will not be described here.

Alternatively, cooling may be provided through an expanded configuration of balloon electrode 204, which may present a partial occlusion at the ablation site, adapted to increase the velocity of blood flowing past the occlusion site, increasing heat transfer provided by the blood flow.

Suitable materials for the balloon electrode 204 may include a material permeable to RF (radio frequency) energy. Further, hydrophilic polymers such as Pebax, nylons, polyesters, or block copolymers. Pebax grades that may be suitable include Pebax MV1074, Pebax MV 1041, Pebax MP 1878, Pebax MV-3000, and Pebax MH-1657. In some embodiments, one or more of the hydrophilic polymers, such as the Pebax grades, may be used in blends with other polymers used for the balloon electrode 204 such as Pebax 6333, Pebax 7033, Pebax 7233, Nylon 12, Vestamid L2101F, Grilamid L20, and Grilamid L25. Suitable hydrophilic polymers may exhibit between 6% to 120% hydrophilicity (or water absorption), between 20% to 50% hydrophilicity, or between other suitable range.

Manufacturing such balloon electrodes could be accomplished in several different ways. As an example, a non-conductive balloon manufactured through blow molding could be applied with a conductive layer through vapor-deposition of gold or platinum, a printed circuit layer, etc. An insulating layer can be further provided that includes windows or regions lacking the insulating layer where the electrode is desired. Further, the conductive layer proximal the electrode may be connected to the power source and used as part of the conductive pathway to the electrode. Alternatively, a masking layer could be applied where the electrode material is not desired. Later, an application of the conductive layer to the balloon may subsequently remove the masking material, which leaves the conductive layer only on portions where required.

As noted above, moving the ablation member 100 and 200 between the expanded and collapsed positions may be effected through the use of an actuation element such as a pull wire referred to as the element 114 and/or the introduction or withdrawal of an inflation medium.

FIG. 3A depicts yet another embodiment of an illustrative the ablation member 300 for use with a nerve modulation system. In the illustrative embodiment, the ablation member 300 may be similar to the ablation member 200 discussed in connection with FIG. 2, with the exception that the function of the nonconductive braid member 102 is replaced by a plurality of nonconductive bumps or protrusions 308 configured to space the electrode 206 from the blood vessel wall during operation. In the illustrated embodiment, the nonconductive bumps 308 are disposed circumferentially around the balloon distally and proximally of the electrode 206. However, it is contemplated that the nonconductive protrusions 308 may be positioned in any manner desired. For example, the protrusions 308 may be positioned on only the proximal or distal side of the electrode or may extend only around a portion of the circumference of the balloon.

As is understood through the earlier description of such electrodes 206, the bumpy ablation member 300 may include one or more electroconductive portions or electrodes 206 as well. For example, while FIG. 3A illustrates a single electroconductive portion 206, it is contemplated that the ablation member 300 may include more than one electroconductive portion 310, as shown in FIG. 3B. As shown in FIG. 3B, the ablation member 300′ may include a plurality of electroconductive portion 310 extending around the circumference of the balloon 204. Each portion 206, 310 may be configured to be conductive, employing any of the various techniques discussed above, such as hydrophilic portions or by printed metal patches or electroconductive strips.

Further, the series of bumps 308, as discussed above, may be disposed on strips 302 and 304, as depicted. In some embodiments, the number, design, spacing, and shape of such strips and/or the bumps 308 may be varied. It may be preferred to arrange electrodes symmetrically, so that the bumps 308 make contact with the blood vessel wall in a patterned manner. Alternatively, it may be preferred to arrange bumps 308 in a random fashion, distributing the stress applied to the blood vessel wall. In yet further embodiments, electrode portions 206, 310 may be disposed even within the regions disposed between the bumps 308. Further embodiments may enable the balloon electrode 204 to have two electrode portions, such as the portion 206, making the balloon electrode 204 a bi-polar electrode, or allowing the balloon electrode 204 to function in a bi-polar mode.

The end sections 306 disposed on either sides of the bumpy ablation member 300 may be similar to the insulated ends 208 (FIG. 2) need not be configured as electrically conductive. Further, the bumps 308, being insulated, may either include insulation coatings, or they may be manufactured from an electrically nonconductive or an insulating material.

The formation of bumps 308 may be in such a manner that an expansion and a collapse of the balloon electrode 204 would sufficiently enable the bumps 308 to expand and contract correspondingly during an application and deployment. In some embodiments, the bumps 308 may be formed as a portion of the balloon 204 and inflated and deflated with the balloon 204 thus having a lower loaded profile. In other embodiments, the bumps 308 may be formed separately having a generally solid configuration and subsequently attached to the balloon surface thus having a higher loaded profile.

It will be understood that embodiments, alternatives, and manufacturing techniques, described for the ablation member 200 may be applicable for the ablation member 300 as well.

In particular, the manufacture of the bumpy ablation member 300 may comprise a blow molding procedure to include forming the initial balloon structure incorporated with the bumps 308. Conductive stripes, printings, etc., may be fabricated later through techniques already discussed in the disclosure. Such forming methodology may further include other technologies well known to the skilled in the art.

The following disclosure contains descriptions in connection with FIG. 4 and FIG. 5, depicting a renal nerve ablation system 400. For ease of understanding, this description includes references for the ablation member 200. It will be understood, however, that the system 400 will function similarly when any of the disclosed ablation members, namely, 100, 200, and 300, are employed.

Accordingly, FIG. 4 is a schematic view of an illustrative renal nerve ablation system 400 in situ within a human body. The system 400 may include one or more electrical conductors 404 for providing conductive power to the ablation member 200 (shown in FIG. 2), disposed at a distal end of the guide sheath 111. A proximal end of the conductor 404, at the proximal end of the elongate device 113, may be connected to the control unit 402, which supplies the required electrical energy to activate the ablation member 200, comprising the balloon electrode 204, at or near a distal end of the elongate device 113. In some instances, return electrode ground pads 406 may be supplied on the legs or at another conventional location on the patient's body to complete the circuit. The control unit 402 may monitor parameters such as power, temperature at the treatment site, voltage, amperage, impedance, pulse size and/or shape and other suitable parameters as well as suitable controls for performing the desired procedure. It will be understood that appropriate sensors such as thermocouples or thermistors may be included at appropriate locations on the system. For example, a thermocouple may be provided proximate the electrode in a system. The ablation member 200 may be configured to operate at a frequency of about 460 kHz. It is contemplated that any desired frequency in the RF range may be used, for example, from 400-500 kHz. However, it is contemplated that frequencies outside the RF spectrum may be used as desired.

FIG. 5 is a further detailed view 500 of the system 400, having the ablation member 200 employed in an expanded position within a blood vessel 510. The guide sheath enters the blood vessel 510 through a bodily opening such as an incision. The system 400 may be deployed from the guide sheath or from a separate retractable sheath that is extended distally from within the guide sheath. The separate retractable sheath may include a self-expanding distal end portion to help deploy the device. The figure depicts the distal end region 512 of the catheter and distal end 518 of the control wire(s) as well. Renal nerves are located on or near an outer surface of the blood vessel 510. In particular, the ablation member 200 is illustrated as touching a blood vessel inner wall 516, keeping the internally disposed balloon electrode(s) centered, at a distance, and out of contact with the inner wall 516 of the blood vessel 510, thereby reducing or possibly eliminating any possible damages to the surrounding tissue. Further, it is understood that changes in blood pressure caused because of an introduction of the nerve modulation assembly, depicted as the system 400 in FIG. 4, and pressure changes caused particularly because of an expansion of the balloon electrode 204 would be kept at a minimum.

The introduction and deployment of the system 400, including an expansion of either of the employed ablation members 100, 200, or 300, either through the pull wire or through an inflation may either be configured through the control unit 402, or may be accomplished manually as well.

Those skilled in the art will recognize that the present disclosure may be manifested in a variety of forms other than the specific embodiments described and contemplated herein. Accordingly, departure in forma and detail may be made without departing from the scope and spirit of the present disclosure as described in the appended claims.

What is claimed is:

1. A nerve modulation assembly, comprising:
an elongate device extending along a central elongate axis, the elongate device having a proximal end and a distal end;
an ablation member disposed at the distal end of the elongate device, and configured to be actuated from the proximal end of the elongate device, the ablation member including:
a radiofrequency based electrode member; and
a spacing member disposed circumferentially around the radiofrequency based electrode member.

2. The assembly of claim 1, wherein an actuation of the electrode member, through the elongate device, is enabled through a radiofrequency based control unit disposed at the proximal end of the elongate device.

3. The assembly of claim 1, wherein the radiofrequency based electrode member is at least one of a balloon electrode, a stent electrode or a slotted tube electrode.

4. The assembly of claim 1, wherein the spacing member is at least one of a nonconductive braid member, a slotted tube or one or more of a series of nonconductive bumps.

5. The assembly of claim 4, wherein the nonconductive braid member includes a plurality of loosely braided spiral coils.

6. The assembly of claim 1, wherein the ablation member is configured to switch between a collapsed position and an expanded position, such that a portion of the spacing member contacts an inner wall of a blood vessel while in the expanded position, keeping the electrode member at a distance from the inner wall.

7. The assembly of claim 6, wherein the collapsed and the expanded position of the ablation member is enabled through a pull wire.

8. The assembly of claim 1, further comprising a guide sheath having a proximal end, a distal end, and a lumen extending therebetween.

9. The assembly of claim 8, wherein the guide sheath is retractable.

10. A renal nerve ablation system, comprising:
a guide sheath having a proximal end, a distal end, and a lumen extending therebetween;
an elongate device extending along a central elongate axis within the lumen, the elongate device having a proximal end and a distal end;
an ablation member disposed at the distal end of the elongate device, the ablation member including:
a radio frequency based electrode member; and
a spacing member disposed circumferentially around the electrode member, wherein the spacing member is configured at least as one of a nonconductive braid member or as one or more of a series of nonconductive bumps, the spacing member configured to contact an inner wall of a blood vessel, while enabling the electrode member to avoid contact with the inner wall of the blood vessel.

11. The system of claim 10, wherein an actuation of the electrode member, through the elongate device, is enabled through a radio frequency based control unit disposed at the proximal end of the elongate device.

12. The system of claim 10, wherein the nonconductive braid member includes a plurality of loosely braided spiral coils.

13. The system of claim 10, wherein the radio frequency based electrode member is at least one of a balloon electrode or a slotted tube electrode.

14. The system of claim 10, wherein the ablation member is configured to switch between a collapsed position and an expanded position, such that the contact of the spacing member to the inner wall is enabled in the expanded position.

15. The system of claim 14, wherein the collapsed and the expanded position of the ablation member is enabled through a pull wire.

16. A method for ablating a nerve through a blood vessel, the method comprising:
advancing a guide sheath, to a desired location within the blood vessel, the guide sheath including a proximal end and a distal end, having an ablation member at the distal end, the ablation member having a radio frequency based electrode member and a spacing member disposed circumferentially around the electrode member;
deploying the ablation member at the desired location within the blood vessel;
using the ablation member such that portions of the spacing member contacts an inner wall of the blood vessel, while enabling the electrode member to avoid a direct contact with the inner wall of the blood vessel; and
activating the electrode member to ablate a desired portion within the blood vessel.

17. The method of claim 16, wherein activating the electrode member is enabled through a radio frequency based control unit disposed at the proximal end of the guide sheath.

18. The method of claim 16, wherein the radio frequency based electrode member is configured at least as a balloon catheter or as a ribbon based slotted tube electrode.

19. The method of claim 16, wherein the spacing member is at least a nonconductive braid member or a series of nonconductive bumps.

20. The method of claim 19, wherein the nonconductive braid member includes a plurality of loosely braided spiral coils.
